(19) United States
(12) Patent Application Publication
(10) Pub. No.: US 2025/0206852 A1
(43) Pub. Date: Jun. 26, 2025

(54) PREPARATION METHOD FOR LOW-TEMPERATURE SOL-AGAR

(71) Applicant: Jimei University, Xiamen (CN)

(72) Inventors: Zedong JIANG, Xiamen (CN); Xin TIAN, Xiamen (CN); Hui NI, Xiamen (CN); Mingjing ZHENG, Xiamen (CN); Tao HONG, Xiamen (CN); Ling WU, Xiamen (CN); Yang HU, Xiamen (CN); Yanbing ZHU, Xiamen (CN); Zhipeng LI, Xiamen (CN); Yuanfan YANG, Xiamen (CN)

(73) Assignee: Jimei University, Xiamen (CN)

(21) Appl. No.: 19/021,340

(22) Filed: Jan. 15, 2025

Related U.S. Application Data
(63) Continuation of application No. PCT/CN2024/130255, filed on Nov. 6, 2024.

(30) Foreign Application Priority Data
Dec. 20, 2023 (CN) 202311771529.9

(51) Int. Cl. C08B 37/00; C12P 19/04

(52) U.S. Cl. CPC C08B 37/00; C08B 37/0003; C12P 19/04

(57) ABSTRACT

A preparation method for low-temperature sol-agar is provided, including the following steps: Gracilaria after microbial fermentation is subjected to alkaline treatment, cleaning, two steps of acidification and washing, washing, bleaching, gel boiling, dehydration, crushing, screw extrusion, microwave drying, etc., to prepare low-temperature sol-agar. The preparation method for low-temperature sol-agar is adopted in the present invention, an agar product is extracted and modified by a microbial fermentation-assisted alkaline method, and the low-temperature sol-agar is prepared by further combining a thermal denaturation processing technology, which not only improves an application performance of the agar product, but also improves the commercial value of the product, reduces the use of conventional process lye while reducing the production cost, which is green and environmentally friendly.

### CROSS REFERENCE TO THE RELATED APPLICATIONS

This application is a continuation application of International Application No. PCT/CN2024/130255, filed on Nov. 6, 2024, which is based upon and claims priority to Chinese Patent Application No. 202311771529.9, filed on Dec. 20, 2023, the entire contents of which are incorporated herein by reference.

### TECHNICAL FIELD

The present invention relates to the field of agar processing technology, in particular to a preparation method for low-temperature sol-agar.

### BACKGROUND

Agar is a functional polysaccharide extracted from red algae (mainly Gracilaria), which is a macromolecular polysaccharide formed by cross-linking of repeated β-D-galactose and α-3,6-anhydro-L-galactose. Agar is a white or light yellow, odorless substance known for its high gel strength, exceptional transparency, and remarkable elasticity and tenacity. It exhibits hydrophilic properties and is widely utilized in food processing, medicine, and bioengineering.

At present, agar applied in the food additive or biomedical material requires a temperature of 95-100° C. for more than 20 minutes to achieve complete dissolution. The heating process for dissolving agar necessitates both high temperatures and extended durations. Furthermore, agar with high gel strength exhibits poor dispersion and diffusion characteristics, leading to uneven dissolution. Additionally, due to its solubility being attainable only at elevated temperatures, agar is not suitable for use as a packaging or matrix material for heat-sensitive active substances in food products. These limitations hinder the broader application of agar within the food and medicine industry.

Low-temperature sol-agar is a high-value deep-processing product that can be rapidly dissolved at a low temperature by modifying the agar raw material to open the hydrogen bond of the agar chain. Due to the destruction of the double helix structure of agar during processing, the gel strength of low-temperature sol-agar is lower than the gel strength of agar. Low-temperature sol-agar also has the characteristics of high transparency, low viscosity and high purity. The sol temperature of low-temperature sol-agar is generally below 85° C., and the sol time varies from a few minutes to more than ten minutes, which is easier to sol than agar.

At present, the preparation methods for low-temperature sol-agar include physical methods, chemical methods and biological enzymatic methods. The physical method is to destroy the hydrogen bonds between agar molecules, increase the solubility of agar in water, and reduce the sol temperature of agar by adding cosolvents or physical processing such as ball milling and spray drying. However, the effect of this physical modification is not sufficient, and the improvement effect is not significant. The chemical method promotes low-temperature sol by adding chemical reagents to change the structure of agar, but its efficiency is low. Meanwhile, it lacks the process of removing chemical reagents, so the safety in the production process is difficult to control. Furthermore, agar with added chemical reagents cannot be applied to food. The biological enzyme method refers to the introduction of enzyme reagents to treat agar in the process of agar production, which breaks the molecular bonds between agar and promotes its degradation, still, the method is costly, and the enzymes involved in the reaction cannot be reused and mass production cannot be achieved.

In conclusion, there is currently a lack of a low-temperature sol-agar production process that meets the criteria of low production costs, sufficient reaction efficiency, absence of new substance introduction, simplicity in operation, and high safety standards while also enabling large-scale production.

Fermentation possesses the unique ability to inhibit the growth of spoilage bacteria and common pathogens, while simultaneously enhancing the nutritional value of food. Additionally, it alters the color, flavor, and texture of the food. The application of probiotics in fermenting raw food materials can disrupt cell walls and facilitate the release of polysaccharides. Furthermore, various enzymes produced during microbial fermentation can biologically modify these polysaccharides, thereby increasing their potential for diverse applications.

### SUMMARY

An objective of the present invention is to provide a preparation method for low-temperature sol-agar, an agar product is extracted and modified by a microbial fermentation-assisted alkaline method, and the low-temperature sol-agar is prepared by further combining a thermal denaturation processing technology, which not only improves an application performance of the agar product, but also improves the commercial value of the product, reduces the use of conventional process lye while reducing the production cost, which is green and environmentally friendly.

To achieve the above objective, the present invention provides a preparation method for low-temperature sol-agar, including the following steps:
S1, performing a microbial fermentation on Gracilaria;
S2, performing an alkaline treatment and washing until neutral for the fermented Gracilaria obtained in step S1;
S3, performing one-step acidification and washing until neutral for the neutral fermented Gracilaria obtained in step S2;
S4, performing two-step acidification and washing until neutral for the neutral one-step acidified Gracilaria obtained in step S3;
S5, bleaching and washing until neutral for the neutral two-step acidified Gracilaria obtained in step S4;
S6, performing a water bath for the neutral bleached Gracilaria obtained in step S5, collecting supernatant and cooling at room temperature to obtain a Gracilaria agar gel;
S7, dividing the Gracilaria agar gel into pieces, packing, freezing and dehydrating and then drying to a constant weight, and crushing and sieving the dried Gracilaria agar;
S8, mixing the agar powder obtained in step S7 with water, obtaining low-temperature sol-agar by screw extrusion, microwave drying, crushing and sieving.

Preferably, the specific steps of microbial fermentation in step S1 are as follows: weighing Gracilaria after washing, impurity removal and drying, adding the Gracilaria to a glucose medium with a mass concentration of 1%-5%, pasteurizing at 65° C. for 30 min, after cooling, inoculating microbial strains at 37° C. under anaerobic conditions for fermentation; a solid-liquid ratio of Gracilaria and glucose medium is 1:(15-80); a solid-liquid ratio of inoculating microbial strains for fermentation is 1:(15-80), an inoculation amount is 1-10%, and fermentation time is 6-36 h.

Preferably, the microbial strains include one of yeast, Lactobacillus, Acetobacter, and Bacillus.

Preferably, the specific steps of alkaline treatment and washing in step S2 are:
S2-1, draining water after the fermentation of Gracilaria, adding NaOH solution with a mass fraction of 1%-5%, and performing a constant temperature treatment at 85° C. for 2-8 h;
S2-2, filtering and separating the fermented Gracilaria from the NaOH solution, and soaking and washing the fermented Gracilaria for 30-90 min with 20-90 times volume of distilled water, and repeating multiple times until a pH of the fermented Gracilaria is 7.

Preferably, the specific steps for one-step acidification in step S3 are:
S3-1, draining water from the fermented Gracilaria washed to neutrality and adding distilled water according to a solid-liquid ratio of 1:(15-80), then adding concentrated sulfuric acid with a volume fraction of 0.043%, standing and soaking for 30-90 min after stirring and mixing well, and discarding an acid washing solution;
S3-2, adding 20-90 times volume of distilled water to the one-step acidified Gracilaria to soak and wash 30-90 min, and repeating multiple times until a pH of the one-step acidified Gracilaria is 7.

Preferably, the specific steps of the two-step acidification and washing in step S4 are:
S4-1, adding oxalic acid with a mass fraction of 0.064%, EDTA-Na₂ with a mass fraction of 0.01267%, and distilled water with a solid-liquid ratio of 1:(15-80) to the neutral one-step acidified Gracilaria into the neutral one-step acidified Gracilaria, standing and soaking for 30-90 min after stirring and mixing well, and discarding the acid washing solution;
S4-2, adding 20-90 times volume of distilled water to the two-step acidified Gracilaria to soak and wash 30-90 min, and repeating multiple times until a pH of the two-step acidified Gracilaria is 7.

Preferably, the specific steps of bleaching and washing in step S5 are: adding a sodium hypochlorite solution with an available chlorine mass fraction of 0.04% to the neutral two-step acidified Gracilaria, standing for 30-90 min after stirring and mixing well, discarding a bleaching solution, and washing the bleached Gracilaria with distilled water to a pH is 7; a solid-liquid ratio of neutral two-step acidified Gracilaria and sodium hypochlorite solution is 1:(15-80).

Preferably, the specific steps of the water bath in step S6 are: adding distilled water to the neutral bleached Gracilaria at a solid-liquid ratio of 1:(15-80), and performing a water bath treatment at 100° C. for 3-8 h, and filtering and separating the algae residue and supernatant.

Preferably, the specific steps of drying and crushing in step S7 are:
S7-1, dividing the Gracilaria agar gel into pieces and packing, freezing for 24-48 h and then taking out, and after thawing at room temperature, filtering and separating water, placing the Gracilaria agar with most of the water removed in an oven, and treating at 60-105° C. for 12-48 h.
S7-2, crushing the dried Gracilaria agar, and screening through an 80-200-mesh standard sieve to obtain Gracilaria agar powder.

Preferably, a moisture content of the agar powder mixed with water in step S8 is 35%.

Therefore, the present invention adopts the above-mentioned preparation method for low-temperature sol-agar, and has the following technical effects:
(1) the agar product was extracted and modified by microbial fermentation-assisted alkaline method, microbial fermentation could soften the cell wall and promote the outflow of pigment and colloidal solution, not only reducing the amount of alkaline solution used in conventional processes, but also degrading the internal structure of colloid;
(2) compared with the low-temperature sol-agar extracted by the conventional alkaline method, the microbial fermentation-assisted alkaline method of the present invention combined with the thermal denaturation processing technology to prepare the low-temperature sol-agar, the yield increases from 7.57% to 10.02%, the sol temperature decreases from 80.93° C. to 69.41° C., and the gel strength decreases from 676.5 g/cm² to 379.3 g/cm²; the low-temperature sol-agar extracted by this process can be widely used in heat-sensitive food and foods for special dietary uses, which improves the application range and economic value of low-temperature sol-agar;
(3) this process not only improves the application performance of agar products, but improves the commercial value of the product, reducing the amount of alkali used in the conventional process while reducing the production cost, and it is green and environmental protection.

Further detailed descriptions of the technical scheme of the present invention can be found in the accompanying drawings and embodiments.

### BRIEF DESCRIPTION OF THE DRAWINGS

To explain the examples of the present invention or the technical solutions in the prior art more clearly, a brief introduction will be made to the accompanying drawings used in the examples or the description of the prior art. It is obvious that the drawings in the following description are only illustrative, and those ordinarily skilled in the art can obtain other embodiment drawings according to these drawings without creative work.

FIG. 1 shows a gel strength, a yield, and a sol temperature of low-temperature sol-agar prepared by embodiments 1-3.

FIG. 2 shows a viscosity, a turbidity and a whiteness of the low-temperature sol-agar prepared by embodiments 1-3;

FIG. 3 shows a sulfate content and a 3,6-anhydrogalactose content of low-temperature sol-agar prepared by embodiments 1-3.

### DETAILED DESCRIPTION OF THE EMBODIMENTS

The technical solution of the present invention will be further elaborated hereafter in conjunction with accompanying drawings and embodiments.

Unless otherwise defined, technical or scientific terms used in the present invention are to be given their ordinary meaning as understood by those of ordinary skill in the art to which the present invention belongs.

### Embodiment 1

The preparation of low-temperature sol-agar by fermentation-assisted alkaline method includes the following steps:
S1, 30 g of Gracilaria after cleaning, impurity removal and drying was accurately weighed, 900 mL of 5% (w/v) glucose medium was added, treated at 65° C. for 30 min for pasteurization, and Lactobacillus fermentation was inoculated at 37° C. under anaerobic conditions after cooled. Where the solid-liquid ratio (w/v) was 1:30, the inoculation amount (v/v) was 2%, and the fermentation time was 12 h.
S2, after fermentation, the water was drained, and 450 mL of 3% (w/v) NaOH solution was added and performed a constant temperature treatment at 85° C. for 3 h. The fermented Gracilaria and lye were filtered and separated with a 200-mesh filter cloth, and the fermented Gracilaria was washed with 20 times the volume of distilled water for 40 min, and repeated multiple times until a pH of the fermented Gracilaria was 7.
S3, the fermented Gracilaria washed to pH=7 was drained and 450 mL of distilled water was added, then 0.195 mL concentrated sulfuric acid was added, stood for 30 min after stirred and mixed well, the acid washing solution was discarded, 20 times the volume of distilled water was added to the one-step acidified Gracilaria, the one-step acidified Gracilaria was soaked and washed for 30 min, repeated multiple times until a pH of the fermented Gracilaria is 7.

0.288 g oxalic acid and 0.057 g EDTA-Na₂ and 450 ml distilled water were added to the one-step acidified Gracilaria, soaked for 30 min after stirred and mixed well, the acid washing solution was discarded, and 20 times the volume of distilled water was added to the two-step acidified Gracilaria, soaked and washed for 30 min, repeated multiple times until a pH of the two-step acidified Gracilaria is 7, the two-step acidification and washing is completed.
S4, a total of 450 mL of sodium hypochlorite solution with an available chlorine mass fraction of 0.04% was added to the two-step acidified Gracilaria which was washed to neutral, stood for 30 min after stirred and mixed well, and the bleaching solution was discarded, the Gracilaria was washed with distilled water to pH=7.
S5, 450 mL of distilled water was added to the bleached Gracilaria which was washed to neutral, and treated in a constant temperature water bath at 100° C. for 3 h, the algae residue and supernatant were filtered and separated, the supernatant was collected and cooled at room temperature to form a gel.
S6, the gelled Gracilaria agar was cut into pieces and packed, and taken after freezing at −20° C. for 24 h, after thawing at room temperature, the water was separated with a 200-mesh filter cloth, the agar that removed most of the water was placed in an oven and treated at 60° C. for 12 h to a constant weight,
S7, the agar that dried to constant weight was crushed with a grinder, and through the 80-mesh standard sieve.
S8, the agar powder was mixed with water, and the moisture content was 35%, after screw extrusion and microwave drying, it was crushed and passed through a 200-mesh sieve to obtain a low-temperature sol-agar product (MLA).

### Embodiment 2

Low-temperature sol-agar was prepared by the conventional alkaline method, including: 30 g of Gracilaria was accurately weighed, 450 mL of 6% (w/v) NaOH solution was added, and the solution was treated at 85° C. for 3 h for the alkaline treatment. The other steps are the same as the embodiment 1, and the low-temperature sol-agar product (HA) is obtained.

### Embodiment 3

Low-temperature sol-agar was prepared by the conventional alkaline method, including: 30 g of Gracilaria was accurately weighed, 450 mL of 3% (w/v) NaOH solution was added, and the solution was treated at 85° C. for 3 h for the alkaline treatment. The other steps are the same as the embodiment 1, and the low-temperature sol-agar product (LA) is obtained.

### Experimental Test

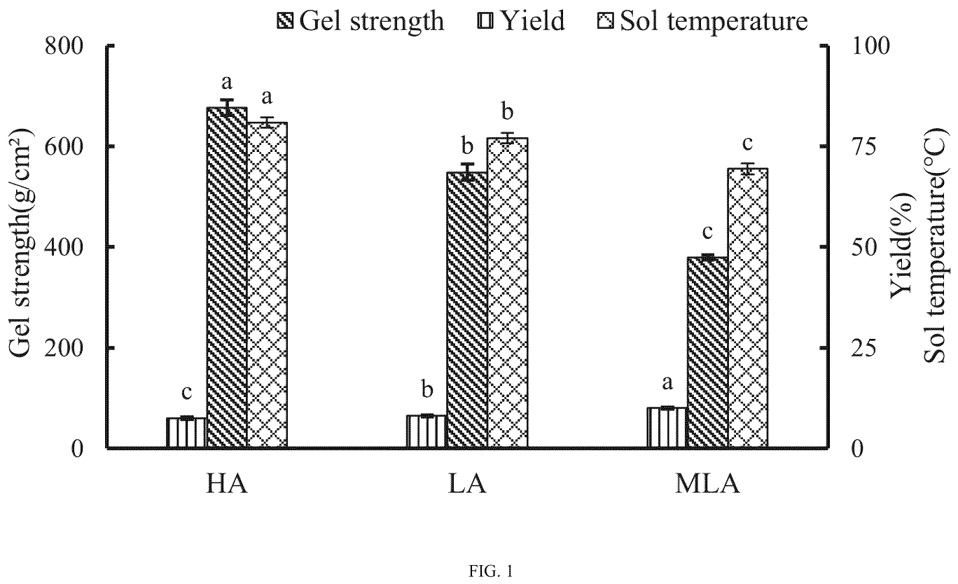

(1) Test the gel strength, yield, and sol temperature of the low-temperature sol-agar products obtained in embodiment 1-3, the results are shown in FIG. 1, the values of a, b, and c in FIG. 1 represent significant differences between different samples (p<0.05). The difference between each index is not significant if there is a same letter, and the difference is significant if there is no same letter.

### 1) Determination of Yield:

The percentage of low-temperature sol-agar powder in the dry weight of Gracilaria is the product yield, and the yield calculation formula is as follows:

$$Yield ( \% ) = \frac{{m}_{1}}{{m}_{2}} \times 100 \%$$

in the formula: m₁ is a mass of low-temperature sol-agar powder/g; m₂ is a mass of dried Gracilaria raw material/g.
   2) Determination of gel strength:

1.5 g of low-temperature sol-agar powder was accurately weighed, added the powder to 98.5 g of distilled water, stirred and mixed well, heated and dissolved in a water bath at 100° C., and the lost water was replenished, after fully dissolved, it was poured into a glass plate with a diameter of 90 mm to cool and solidify, covered with a preservative film, and stood at 20° C. for 12 h.

The plate to be tested was put on the left tray of the tray balance, the plunger with a cross-sectional area of 1 cm² after touching the gel surface was fixed, the beaker was put on the right side of the tray balance, distilled water was poured into the beaker slowly and continuously, and the water can not be interrupted, then pouring water was stopped immediately after breaking the gel surface, and the weight of the beaker and distilled water were recorded at this time, the ratio of the weight of the beaker and distilled water to the cross-sectional area of the plunger is the gel strength of the product:

$$Gel strength ( g / {cm}^{2} ) = \frac{M}{S}$$

in the formula: M is a total weight of distilled water and beaker/g; S is a cross-sectional area of the plunger/cm².

### 3) Determination of Sol Temperature:

A 1.5% low-temperature sol-agar solution was prepared, and 10.0 mL of each solution was poured into three 18×180 mm test tubes and cooled at room temperature overnight. After forming a gel, a small glass bead is placed on the surface of the gel, respectively, and a rubber plug is plugged. Then the test tube was placed in a 30° C. water bath and heated at a rate of 1° C./min, the temperature was recorded when the glass beads fell.

### Results Analysis

The yield of low-temperature sol-agar (HA, LA, MLA) prepared by high alkaline, low alkaline and fermentation low alkaline processes was 7.57%, 8.08% and 10.02%, respectively (p<0.05).

Alkaline treatment can soften the cell wall and promote the outflow of glue and pigment. Under a high alkaline concentration, the cell wall was severely damaged, which promoted the outflow of Gracilaria gel solution, but the subsequent cleaning and neutralization process with clean water caused a large amount of gel solution loss, less gel solution was retained in Gracilaria, and the yield was the least. The damage of low alkaline solution to the cell wall of algae is not as serious as that of high alkaline concentration, but the gel solution can retain the inside of algae due to the less loss of cleaning and neutralization, so the yield of LA is higher than that of HA. The yield of MLA was the highest, due to the destruction of the cell wall of the algae by fermentation and low alkaline solution, which promoted the outflow of gel solution, but the low alkaline solution treated Gracilaria was easier to wash and neutralize than the high alkaline treated Gracilaria, and the loss of gel solution was less, and the yield was the highest.

The gel strength of HA, LA and MLA were 676.5 g/cm², 548.0 g/cm² and 379.3 g/cm², respectively (p<0.05).

The concentration of lye, time and temperature are the main factors affecting the strength of agar gel. The gel strength of HA prepared under high alkaline conditions was higher than that of LA and FLA prepared under low alkaline conditions. Fermentation degraded the internal structure of the colloid, and the gel strength of MLA was the lowest.

The sol temperature of HA, LA and MLA were 80.93° C., 77.05° C. and 69.41° C., respectively. The sol temperature is related to the molecular structure and molecular weight of agar, and the molecular structure density is related to the gel strength. The change trend of sol temperature of HA, LA and MLA is consistent with the change trend of gel strength.

The yield, gel strength and sol temperature of low-temperature sol-agar prepared by fermentation low-alkaline process were 10.02%, 379.3 g/cm² and 69.41° C., respectively, compared with the low-temperature sol-agar prepared under high alkaline conditions (7.57%, 676.5 g/cm², 80.93° C.), the yield was increased, the sol temperature was reduced, and the alkali consumption was saved by 50%.

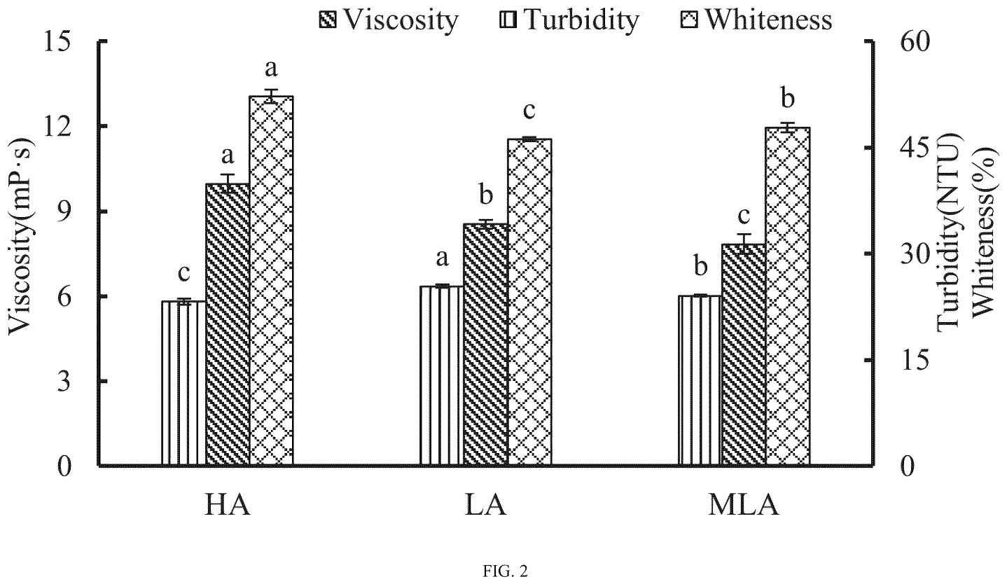

(2) The viscosity, turbidity and whiteness of the low-temperature sol-agar products obtained in embodiment 1-3, the results are shown in FIG. 2, a, b, and c represent significant differences between different samples (p<0.05). The difference between each index is not significant if there is a same letter, and the difference is significant if there is no same letter.

### 1) Determination of Gel Viscosity:

100 mL of low-temperature sol-agar solution with a mass fraction of 1.5% was prepared, after being fully dissolved by heating in a water bath at 100° C., the lost water was replenished, poured into a cuvette at 85° C., and placed in a turbidimeter for recording after the reading was stable.

### 2) Determination of Gel Turbidity:

According to GB 1886.169-2016, 4.5 g of low-temperature sol-agar powder was weighed, about 300 mL of deionized water was added, stirred and mixed well, dissolved in a 100° C. water bath, and the lost water was replenished, after fully dissolved, it was cooled to 77° C. and placed in a 75° C. thermostatic bath. The pendulum and protective sleeve of the viscometer were pre-heated in water to 75° C. and dried, and then mounted on the viscometer, the NDJ-9S digital rotational viscometer was used to measure, the unit is mP·s.

### 3) Determination of Whiteness:

The low-temperature sol-agar powder was passed through an 80-mesh standard sieve and the whiteness was measured by a whiteness meter.

### Results Analysis

The viscosity of HA, LA and MLA were 9.97 mP·s, 8.53 mP·s and 7.83 mP·s, respectively. The viscosity of HA is the largest because of the denser molecular structure of HA. The turbidity of HA, LA and MLA was 23.23 NTU, 25.40 NTU and 24.07 NTU, respectively, and the whiteness of HA, LA and MLA was 52.20%, 46.20% and 46.80%, respectively. Turbidity and whiteness are the evaluation indexes of agar color. The alkaline treatment process can remove Gracilaria pigment and improve the quality of agar. HA had lower turbidity and higher whiteness than LA and MLA, indicating that high alkaline conditions were more likely to remove pigments from algae. The turbidity and whiteness of MLA were between HA and LA, indicating that the fermentation softened the cell wall and also removed some pigments.

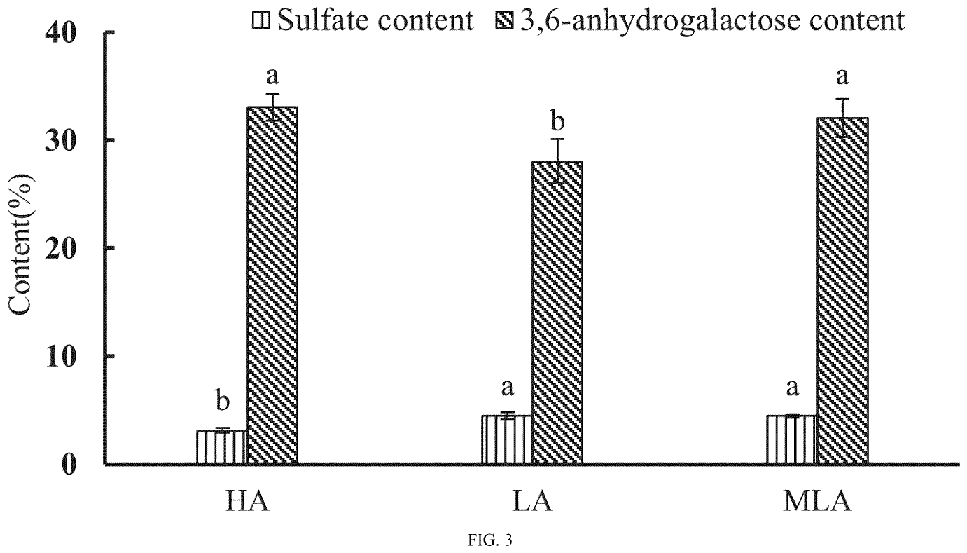

(3) The determination of sulfate content and 3,6-anhydrogalactose content of the low-temperature sol-agar products obtained in embodiment 1-3, the results are shown in FIG. 3, a, b, and c represent significant differences between different samples (p<0.05). The difference between each index is not significant if there is a same letter, and the difference is significant if there is no same letter.

### 1) Determination of Sulfate:

Preparation of 1% Tween-BaCl₂ solution: 1 g of Tween 20 and 1 g BaCl₂ were weighed respectively, and 98 g of water was added, mixed well and filtered to obtain a clear solution for later use.

Preparation of K₂SO₄ standard solution: 0.1088 g (accurate to 0.0001 g) of K₂SO₄ which had been dried to constant weight at 105° C. was weighed, and the solution was diluted to 100 mL with 1.0 mol/L hydrochloric acid and stored for later use.

Digestion of low-temperature sol agar: 0.05 g of sample was weighed in a 25 mL colorimetric tube and 25 mL of 1 mol/L hydrochloric acid was added. Then digested in a water bath at 100° C. for 5 h, after digestion, the digestive juice was cooled to room temperature and diluted to 25 mL, finally, the clear digestive juice was obtained by decolorization and filtration with activated carbon for later use.

The drawing of the standard working curve: the K₂SO₄ standard solution was diluted 5 times, and 0 mL, 0.2 mL, 0.4 mL, 0.6 mL, 0.8 mL, and 1.0 mL of diluents were taken and added to the test tube, and 1.0 mol/L hydrochloric acid was supplemented to 1.0 mL. The 1 mL of sample solution was added with 3 mL of Tween-BaCl₂ solution, shaken and mixed well, and stood for 10 min, the absorbance was measured at a wavelength of 360 nm, and the standard curves of the absorbance of different concentrations of sulfate were obtained.

Determination of sulfate content: 1 mL of digestive juice was taken and 3 mL of Tween-BaCl₂ solution was added, shaken and mixed well, and stood for 10 min, the absorbance was measured at a wavelength of 360 nm, and the sulfate content of the sample was calculated by using the standard working curve.

2) Determination of 3,6-anhydrogalactose:

Preparation of standard solution: fructose was dried to the constant weight, 1 mg was accurately weighed, and diluted to 10 mL volumetric flask with ultrapure water to prepare a standard solution of 100 μg/mL.

Preparation of resorcinol reserve solution: 150 mg of resorcinol was dissolved in distilled water, and diluted to the scale with a 100 mL brown volumetric flask, stored at 4° C., and used within one month.

Preparation of acetal reserve solution: 84 μL of acetal was drawn and 9.916 mL of distilled water was added, mixed well and stored in brown bottles at 4° C. for one month. Before preparing the colorimetric reagent, 1 mL of acetal reserve solution was diluted to 25 mL and used on the same day.

Preparation of working solution (resorcinol-acetal reagent): 100 mL of concentrated hydrochloric acid was slowly added to 9 mL of resorcinol reserve solution, and then 1 mL of diluted acetal reserve solution was added, this solution needs to be prepared on the same day and away from light, and used within 3 h.

The facture of standard curve: 100 μg/mL fructose solution: 0.02 mL, 0.04 mL, 0.08 mL, 0.12 mL, 0.16 mL, 0.24 mL were added to the test tube respectively, and distilled water was added to replenish to 0.4 mL, and 0.4 mL of distilled water was taken as blank. Ice bath for 5 min, 175 rpm, 2 mL of newly configured cold working solution was added to the test tube under ice bath, placed in water bath at 80° C. for 15 min, ice bath for 1.5 min, 175 rpm, and the absorbance was measured at 554 nm wavelength, the standard curve was plotted with the standard concentration as the abscissa and the absorbance as the ordinate.

Sample determination: the samples were prepared into 30 μg/mL of solution with distilled water. 400 μL of the solution was drawn, operated with the above method to measure the absorbance value, and substituted into the standard curve to determine the 3,6-anhydrogalactose content in the sample.

### Results Analysis

The sulfate contents of HA, LA and MLA were 3.20%, 4.59% and 4.58%, respectively, and the contents of 3,6-anhydrogalactose were 33.06%, 28.04% and 32.06%, respectively. Alkaline treatment can improve the gel strength of agar, and the sulfate group will be converted into 3,6-anhydrogalactose; the gel strength of agar was inversely proportional to the content of sulfate and proportional to the content of 3,6-anhydrogalactose.

The sulfate content in HA prepared under high alkaline conditions was lower than the sulfate content in LA and MLA prepared under low alkaline conditions, and the difference in sulfate content between LA and MLA was not significant, indicating that the solubility of alkaline solution affected the sulfate content in low-temperature sol-agar. The difference between the 3,6-anhydrogalactose of MLA and HA was not significant, and the galactose content of MLA and HA was higher than the galactose content of LA, which was due to the combined effect of low alkaline solution and fermentation.

Therefore, a preparation method for low-temperature sol-agar is adopted in the present invention, an agar product is extracted and modified by a microbial fermentation-assisted alkaline method, and the low-temperature sol-agar is prepared by further combining a thermal denaturation processing technology, which not only improves an application performance of the agar product, but also improves the commercial value of the product, reduces the use of conventional process lye while reducing the production cost, which is green and environmentally friendly.

Finally, it should be noted that the above examples are merely used for describing the technical solutions of the present invention, rather than limiting the same. Although the present invention has been described in detail with reference to the preferred examples, those of ordinary skill in the art should understand that the technical solutions of the present invention may still be modified or equivalently replaced. However, these modifications or substitutions should not make the modified technical solutions deviate from the spirit and scope of the technical solutions of the present invention.

What is claimed is:

1. A preparation method for a low-temperature sol-agar, comprising the following steps:
S1, performing a microbial fermentation on Gracilaria to obtain fermented Gracilaria;
S2, performing an alkaline treatment and a washing until neutral for the fermented Gracilaria obtained in the step S1 to obtain neutral fermented Gracilaria;
S3, performing a one-step acidification and a washing until neutral for the neutral fermented Gracilaria obtained in the step S2 to obtain neutral one-step acidified Gracilaria;
S4, performing a two-step acidification and a washing until neutral for the neutral one-step acidified Gracilaria obtained in the step S3 to obtain neutral two-step acidified Gracilaria;
S5, bleaching and washing until neutral the neutral two-step acidified Gracilaria obtained in the step S4 to obtain neutral bleached Gracilaria,
S6, performing a water bath for the neutral bleached Gracilaria obtained in the step S5, and collecting a supernatant and cooling the supernatant at a room temperature to obtain a Gracilaria agar gel;
S7, dividing the Gracilaria agar gel into pieces, packing, freezing and dehydrating, and then drying to a constant weight to obtain a dried Gracilaria agar, and crushing and sieving the dried Gracilaria agar to obtain an agar powder; and
S8, mixing the agar powder obtained in the step S7 with water, obtaining the low-temperature sol-agar by a screw extrusion, a microwave drying, a crushing, and a sieving.

2. The preparation method for the low-temperature sol-agar according to claim 1, wherein steps of the microbial fermentation in the step S1 are as follows:
weighing the Gracilaria after a cleaning, an impurity removal, and a drying, adding the Gracilaria to a glucose medium with a mass concentration of 1%-5%, pasteurizing at 65° C. for 30 min, and after a cooling, inoculating microbial strains at 37° C. under anaerobic conditions for the microbial fermentation; wherein a solid-liquid ratio of the Gracilaria and the glucose medium is 1:(15-80);
wherein the microbial strains comprise one of yeast, Lactobacillus, Acetobacter, and Bacillus; a solid-liquid ratio of inoculating the microbial strains for the microbial fermentation is 1:(15-80), an inoculation amount is 1-10%, and a fermentation time is 6-36 h.

3. The preparation method for the low-temperature sol-agar according to claim 1, wherein steps of the alkaline treatment and the washing in the step S2 are:
S2-1, draining water after the microbial fermentation of the Gracilaria, adding a NaOH solution with a mass fraction of 1%-5%, and performing a constant temperature treatment at 85° C. for 2-8 h;
S2-2, filtering and separating the fermented Gracilaria from the NaOH solution, soaking and washing the fermented Gracilaria for 30-90 min with 20-90 times volume of distilled water, and repeating a plurality of times until a pH of the fermented Gracilaria is 7.

4. The preparation method for the low-temperature sol-agar according to claim 1, wherein steps for the one-step acidification in the step S3 are:
S3-1, draining water from the neutral fermented Gracilaria and adding distilled water according to a solid-liquid ratio of 1:(15-80), then adding concentrated sulfuric acid with a volume fraction of 0.043%, standing and soaking for 30-90 min after stirring and mixing well, and discarding an acid washing solution; and
S3-2, adding 20-90 times volume of the distilled water to one-step acidified Gracilaria to soak and wash 30-90 min, and repeating a plurality of times until a pH of the one-step acidified Gracilaria is 7.

5. The preparation method for the low-temperature sol-agar according to claim 1, wherein steps of the two-step acidification and the washing in the step S4 are:
S4-1, adding oxalic acid with a mass fraction of 0.064%, EDTA-Na₂ with a mass fraction of 0.01267%, and distilled water with a solid-liquid ratio of 1:(15-80) to the neutral one-step acidified Gracilaria into the neutral one-step acidified Gracilaria, standing and soaking for 30-90 min after stirring and mixing well, and discarding an acid washing solution; and
S4-2, adding 20-90 times volume of the distilled water to two-step acidified Gracilaria to soak and wash 30-90 min, and repeating a plurality of times until a pH of the two-step acidified Gracilaria is 7.

6. The preparation method for the low-temperature sol-agar according to claim 1, wherein steps of the bleaching and the washing in the step S5 are: adding a sodium hypochlorite solution with an available chlorine mass fraction of 0.04% to the neutral two-step acidified Gracilaria, standing for 30-90 min after stirring and mixing well, discarding a bleaching solution, and washing bleached Gracilaria with distilled water to a pH of 7; wherein a solid-liquid ratio of the neutral two-step acidified Gracilaria and the sodium hypochlorite solution is 1:(15-80).

7. The preparation method for the low-temperature sol-agar according to claim 1, wherein steps of the water bath in the step S6 are: adding distilled water to the neutral bleached Gracilaria at a solid-liquid ratio of 1:(15-80), performing a water bath treatment at 100° C. for 3-8 h, and filtering and separating an algae residue and the supernatant.

8. The preparation method for the low-temperature sol-agar according to claim 1, wherein steps of the drying and the crushing in the step S7 are:
S7-1, dividing the Gracilaria agar gel into the pieces and packing, freezing for 24-48 h and then taking out, and after a thawing at the room temperature, filtering and separating water, placing a Gracilaria agar with most of the water removed in an oven, and treating at 60-105° C. for 12-48 h; and
S7-2, crushing the dried Gracilaria agar, and screening through an 80-200-mesh standard sieve to obtain the agar powder.

9. The preparation method for the low-temperature sol-agar according to claim 1, wherein a moisture content of the agar powder mixed with the water in the step S8 is 35%.

10. A low-temperature sol-agar prepared by the preparation method according to claim 1.

11. The low-temperature sol-agar according to claim 10, wherein in the preparation method, steps of the microbial fermentation in the step S1 are as follows:
weighing the Gracilaria after a cleaning, an impurity removal and a drying, adding the Gracilaria to a glucose medium with a mass concentration of 1%-5%, pasteurizing at 65° C. for 30 min, and after a cooling, inoculating microbial strains at 37° C. under anaerobic conditions for the microbial fermentation; wherein a solid-liquid ratio of the Gracilaria and the glucose medium is 1:(15-80);
wherein the microbial strains comprise one of yeast, Lactobacillus, Acetobacter, and Bacillus; a solid-liquid ratio of inoculating the microbial strains for the microbial fermentation is 1:(15-80), an inoculation amount is 1-10%, and a fermentation time is 6-36 h.

12. The low-temperature sol-agar according to claim 10, wherein in the preparation method, steps of the alkaline treatment and the washing in the step S2 are:
S2-1, draining water after the microbial fermentation of the Gracilaria, adding a NaOH solution with a mass fraction of 1%-5%, and performing a constant temperature treatment at 85° C. for 2-8 h;
S2-2, filtering and separating the fermented Gracilaria from the NaOH solution, and soaking and washing the fermented Gracilaria for 30-90 min with 20-90 times volume of distilled water, and repeating a plurality of times until a pH of the fermented Gracilaria is 7.

13. The low-temperature sol-agar according to claim 10, wherein in the preparation method, steps for the one-step acidification in the step S3 are:
S3-1, draining water from the neutral fermented Gracilaria and adding distilled water according to a solid-liquid ratio of 1:(15-80), then adding concentrated sulfuric acid with a volume fraction of 0.043%, standing and soaking for 30-90 min after stirring and mixing well, and discarding an acid washing solution; and
S3-2, adding 20-90 times volume of the distilled water to one-step acidified Gracilaria to soak and wash 30-90 min, and repeating a plurality of times until a pH of the one-step acidified Gracilaria is 7.

14. The low-temperature sol-agar according to claim 10, wherein in the preparation method, steps of the two-step acidification and the washing in the step S4 are:
S4-1, adding oxalic acid with a mass fraction of 0.064%, EDTA-Na₂ with a mass fraction of 0.01267%, and distilled water with a solid-liquid ratio of 1:(15-80) to the neutral one-step acidified Gracilaria into the neutral one-step acidified Gracilaria, standing and soaking for 30-90 min after stirring and mixing well, and discarding an acid washing solution; and
S4-2, adding 20-90 times volume of the distilled water to two-step acidified Gracilaria to soak and wash 30-90 min, and repeating a plurality of times until a pH of the two-step acidified Gracilaria is 7.

15. The low-temperature sol-agar according to claim 10, wherein in the preparation method, steps of the bleaching and the washing in the step S5 are: adding a sodium hypochlorite solution with an available chlorine mass fraction of 0.04% to the neutral two-step acidified Gracilaria, standing for 30-90 min after stirring and mixing well, discarding a bleaching solution, and washing bleached Gracilaria with distilled water to a pH of 7; wherein a solid-liquid ratio of the neutral two-step acidified Gracilaria and the sodium hypochlorite solution is 1:(15-80).

16. The low-temperature sol-agar according to claim 10, wherein in the preparation method, steps of the water bath in the step S6 are: adding distilled water to the neutral bleached Gracilaria at a solid-liquid ratio of 1:(15-80), and performing a water bath treatment at 100° C. for 3-8 h, and filtering and separating an algae residue and the supernatant.

17. The low-temperature sol-agar according to claim 10, wherein in the preparation method, steps of the drying and the crushing in the step S7 are:
S7-1, dividing the Gracilaria agar gel into the pieces and packing, freezing for 24-48 h and then taking out, and after a thawing at the room temperature, filtering and separating water, placing a Gracilaria agar with most of the water removed in an oven, and treating at 60-105° C. for 12-48 h; and
S7-2, crushing the dried Gracilaria agar, and screening through an 80-200-mesh standard sieve to obtain the agar powder.

18. The low-temperature sol-agar according to claim 10, wherein in the preparation method, a moisture content of the agar powder mixed with the water in the step S8 is 35%.
